(19) United States
(12) Patent Application Publication
(10) Pub. No.: US 2022/0192689 A1
(43) Pub. Date: Jun. 23, 2022

(54) Lesion Crossing Device with Embolic Protection

(71) Applicant: Fortuna Clinic, LLC, Birmingham, AL (US)

(72) Inventor: Jan Skowronski, Birmingham, AL (US)

(73) Assignee: Fortuna Clinic, LLC, Birmingham, AL (US)

(21) Appl. No.: 17/556,967

(22) Filed: Dec. 20, 2021

Related U.S. Application Data
(60) Provisional application No. 63/128,593, filed on Dec. 21, 2020.

(51) Int. Cl. A61B 17/221

(52) U.S. Cl. CPC A61B 17/221; A61B 2017/2217; A61B 2017/2215; A61B 2017/2212

(57) ABSTRACT

The present disclosure includes apparatuses and methods for a distal protection system. The system may include a delivery catheter, an embolic protection apparatus, and a retrieval catheter. An improved embolic protection apparatus with a smaller diameter wire in the area of the filter is also disclosed. Embolic protection apparatuses that can be used in a wide diameter range of lumens is disclosed.

This application claims the benefit of U.S. Patent Application No. 63/128,593, filed Dec. 21, 2020, the entire content of which is hereby incorporated herein by reference.

### TECHNICAL FIELD

This application is directed to devices, systems, and methods for treating lesions, including crossing narrow passages of lumen segments or total occlusions and devices, systems, and methods providing embolic protection.

### BACKGROUND

Peripheral artery disease (PAD) and coronary artery disease (CAD) affect millions of people in the United States alone. PAD and CAD are silent, dangerous diseases that can have catastrophic consequences when left untreated. CAD is the leading cause of death for in the United States while PAD is the leading cause of amputation in patients.

Coronary artery disease (CAD) and Peripheral artery disease (PAD) are both caused by the progressive narrowing of the blood vessels most often caused by atherosclerosis, the collection of plaque or a fatty substance along the inner lining of the artery wall. Over time, this substance hardens and thickens, which may interfere with blood circulation to the arms, legs, stomach and kidneys. This narrowing forms a lesion, completely or partially restricting flow through the artery. Blood circulation to the brain and heart may be reduced, increasing the risk for stroke and heart disease. A percentage of the population has arterial atherosclerosis that totally occludes portions of the patient's vasculature and presents significant risk to the patient's health. For example, in cases of severe or chronic total occlusions (CTOs) of a coronary artery, the result may be painful angina, loss of functional cardiac tissue or death. In another example, complete occlusion of the arteries in the leg may result in critical limb ischemia and subsequent limb amputation. Another mechanism that leads to limited flow to the limbs is called Acute Limb Ischemia (ALI) and is caused by a blood clot, either forming within the blood vessel, or more often traveling from the heart to the limb and causing acute cessation of blood flow to the limb. This mechanism is considered medical emergency, and if the blood flow is not restored within hours, it will lead to limb amputation.

Commonly known endovascular devices and techniques for the treatment of chronic total occlusions (CTOs) are either inefficient (resulting in a time-consuming procedure), very expensive, or have a high risk of perforating a vessel (resulting in an unsafe procedure) or fail to cross the lesion (resulting in poor efficacy). Bypass surgery is often the preferred treatment for patients with chronic total occlusions both in the heart and peripheral arteries, but surgical procedures are undesirably invasive, and associated with high level of mortality and morbidity, as well as prolonged hospitalization.

There are a number of products on the market that are designed specifically for crossing CTOs and these can be categorized as either intraluminal, subintimal, or re-entry devices. Intraluminal crossing will produce the dissection plane of a long occlusive lesion, protect collaterals and keep treatment options open. Subintimal crossing may require “re-entry” back to the true lumen beyond the occluded segment, putting collaterals at risk and limiting treatment options. It may also increase the rates of complications such as perforation and dissection and extend procedure time with resultant increased radiation and contrast exposure. Also, below the knee, once a wire has crossed into the adventitia it is extremely difficult to re-enter the true lumen.

Once a physician has crossed the CTO, an endovascular procedure may be performed to treat the occluded lumen. The procedures to treat occlusive vascular diseases, such as angioplasty, atherectomy and stent placement, often cause blood clots to form and/or atheromatous material to dislodge from inside the vessel walls and enter the bloodstream. The dislodged material (e.g., plaque), known as at atheroemboli, may be large enough to occlude downstream vessels, potentially blocking blood flow to tissues. Additionally, the blood clots, known as thromboemboli, may be large enough to block the blood flow downstream.

There are numerous previously known interventional systems and methods that employ a filter mechanism designed to capture material dislodged from vessel walls during the treatment or diagnosis of vascular disease. Many of the more recent devices employ an expandable filter disposed at the distal end of a guide wire. These filters have various configurations, such as mesh or microporous membranes in the form of sleeves, parachutes or baskets attached to the guide wire or other delivery mechanism by means of struts, wires, ribs or frames. The meshes are frequently made of woven or braided fibers or wires made of stainless steel, nitinol, platinum alloy, polyester, nylon or porous plastics, for example. The microporous membranes are typically made of a polymer material such as polypropylene, polyurethane, polyester, polyethylene terephthalate, polytetrafluoroethylene or combinations thereof.

A lesion crossing catheter device designed to address some of these concerns and an embolic filter are described herein.

### SUMMARY OF THE DISCLOSURE

Described herein are lesion-crossing devices that address the concerns of the prior art devices and embolic protection systems deployed in a body vessel or cavity for the collection of loosened and/or dislodged debris.

In one embodiment, the lesion crossing device comprises a delivery catheter that contains a wire based embolic protection device. When crossing the lesion, the wire is extended out the distal end of the catheter such that it loops back toward the proximal end. This looped wire aids in crossing the lesion, and will tend to stay intraluminal, minimizing the chance of migration into the adventitia or beyond.

In another embodiment, the embolic protection device comprises an expandable loop with a filter attached to a guidewire. The section of wire proximal of the expandable loop is of a diameter that allows for improved support for delivery during endovascular procedures. In some embodiments, it has a diameter of 0.035 inches. The wire distal of the expandable loop is the same or a smaller diameter than the proximal section of the wire. The wire at the basket section may be of smaller diameter to allow filter to more easily fit into the delivery catheter, after folding or compressing the filter. This allows for a smaller collapsed cross-sectional area of the embolic filter such that a smaller diameter delivery catheter can be used. In some embodiments, the wire distal of the basket has a larger diameter than the wire from the expandable loop to the distal end of the porous filter. In some embodiments, the wire distal of the basket has a smaller diameter than the wire from the expandable loop to the distal end of the porous filter.

In another embodiment, a CTO crossing device comprising a delivery catheter and a wire embolic protection device is used. With the distal end of the wire extending past the distal end of the catheter and looping back toward the proximal end of the catheter, the physician advances the system through the lesion. Once across the lesion, the delivery catheter is withdrawn, and the embolic filter expands. In this state, the expandable loop contacts the lumen wall. After the delivery catheter is withdrawn, the physician may perform a procedure at the site of the lesion. If the lesion is in a vascular lumen, an endovascular procedure may be performed. If the lesion is in a non-vascular lumen, an endoscopic procedure may be performed. The procedure may include balloon catheters, drug coated balloon catheters, stent delivery catheters, drug coated stent delivery catheters, thrombectomy catheters, and atherectomy systems. In some embodiments, after the procedure is completed, the physician may advance a retrieval catheter over the wire to collapse the expandable loop of the embolic protection device to allow for the removal of the system.

In another embodiment, the delivery catheter is sized to fit within a retrieval catheter. If the physician needs additional support when crossing the lesion with the delivery catheter system, the retrieval catheter can be advanced over the delivery catheter/wire filter system to provide additional backup support. In other embodiments, a smaller sized retrieval catheter is used such that the delivery catheter is not sized to fit within the retrieval catheter.

In another embodiment, a scaffold is placed within the filter. The scaffold helps maintain the filter in an expanded configuration, especially in small diameter lumens. The scaffold may comprise one or more metallic loops.

In some embodiments, a device comprises an embolic protection apparatus comprising a wire and a basket attached to the wire. The basket comprises a structural loop attached to a porous filter. The wire comprises a first portion and a second portion, wherein the first portion is proximal to the basket and the second portion is coextensive with at least a portion of the basket. A cross-sectional area of the first portion is greater than a cross-sectional area of the second portion.

In some embodiments, the device further comprises a catheter comprising a lumen and the basket is positioned in the lumen. In some embodiments, the wire comprises a third portion distal to the basket. In some embodiments, the third portion extends distally from the catheter, comprises a bend and overlaps the catheter. In some embodiments, a cross-sectional area of the third portion is greater than the cross-sectional area of the second portion.

In some embodiments, the basket comprises a first basket and the embolic protection apparatus comprises a second basket attached to the wire. In some embodiments, the second basket is larger than the first basket.

In some embodiments, a basket comprises a scaffold arranged to support the porous filter. In some embodiments, the scaffold is attached to the wire.

In some embodiments, the structural loop is variable in size. In some embodiments, a structural loop comprises a secondary loop.

In some embodiments, a device comprises an embolic protection apparatus comprising a wire, a first basket attached to the wire and a second basket attached to the wire. The first basket comprises a first loop attached to a first porous filter. The second basket comprises a second loop attached to a second porous filter. In some embodiments, the second basket is larger than the first basket. In some embodiments, an aperture defined by the second loop is larger than an aperture defined by the first loop.

In some embodiments, the second loop comprises a secondary loop.

In some embodiments, the device comprises a catheter comprising a lumen, the first basket is positioned in the lumen and the second basket is positioned in the lumen.

In some embodiments, a device comprises an embolic protection apparatus comprising a wire and a basket attached to the wire. The basket comprises a structural loop attached to a porous filter and the structural loop comprises a secondary loop. In some embodiments, wherein a cross-sectional area of the structural loop changes as a size of the secondary loop changes. In some embodiments, the structural loop is attached to the wire at an attachment point and the secondary loop located opposite the attachment point. In some embodiments, the wire comprises a first portion and a second portion, the first portion proximal to the basket and the second portion coextensive with at least a portion of the basket. A cross-sectional area of the first portion greater than a cross-sectional area of the second portion.

### BRIEF DESCRIPTION OF THE DRAWINGS

FIG. 1A is a schematic diagram of a delivery catheter in accordance with a number of embodiments of the present disclosure.

FIG. 1B is a schematic diagram of a retrieval catheter in accordance with a number of embodiments of the present disclosure.

FIG. 1C is a schematic diagram of an embolic protection apparatus in accordance with a number of embodiments of the present disclosure.

FIG. 1D is a schematic diagram of an embolic protection apparatus positioned within the delivery catheter in accordance with a number of embodiments of the present disclosure.

FIGS. 2A to 2G show an embodiment of the invention used in a lesion in a blood vessel.

FIG. 3 shows an embodiment of the invention where the retrieval catheter is used with the delivery catheter to provide backup support for the delivery catheter and embolic protection apparatus.

FIGS. 4A and 4B show an embodiment of the invention used in two different diameter blood vessels.

FIG. 5 shows an embodiment of the embolic protection apparatus in accordance with a number of embodiments of the present disclosure.

FIGS. 6A and 6B show an embodiment of the embolic protection apparatus in accordance with a number of embodiments of the present disclosure.

### DETAILED DESCRIPTION

The present disclosure includes methods and apparatuses for devices for crossing lesions and for providing embolic protection. An example apparatus includes delivery catheter sized to contain an embolic protection apparatus. The delivery catheter is used to cross lesions. In some examples, the distal end of the wire is positioned outside the distal end of the delivery catheter when crossing the lesion. In some examples, a retrieval catheter is used to capture the expandable distal protection apparatus for removal from the body.

In the following detailed description of the present disclosure, reference is made to the accompanying drawings that form a part hereof, and in which is shown by way of illustration how one or more embodiments of the disclosure may be practiced. These embodiments are described in sufficient detail to enable those of ordinary skill in the art to practice the embodiments of this disclosure, and it is to be understood that other embodiments may be utilized and that process, electrical, and structural changes may be made without departing from the scope of the present disclosure.

As used herein, designators such as “X”, “Y”, “N”, “M”, etc., particularly with respect to reference numerals in the drawings, indicate that a number of the particular feature so designated can be included. It is also to be understood that the terminology used herein is for the purpose of describing particular embodiments only and is not intended to be limiting. As used herein, the singular forms “a”, “an”, and “the” can include both singular and plural referents, unless the context clearly dictates otherwise. In addition, “a number of”, “at least one”, and “one or more” (e.g., a number of pivot points) can refer to one or more pivot points, whereas a “plurality of” is intended to refer to more than one of such things. Furthermore, the words “can” and “may” are used throughout this application in a permissive sense (i.e., having the potential to, being able to), not in a mandatory sense (i.e., must). The term “include,” and derivations thereof, means “including, but not limited to”. The terms “coupled” and “coupling” mean to be directly or indirectly connected physically or for access to and movement of the movable handle member, as appropriate to the context.

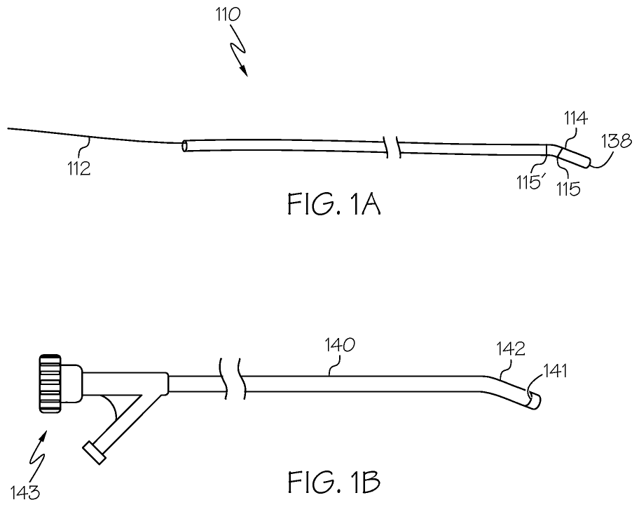

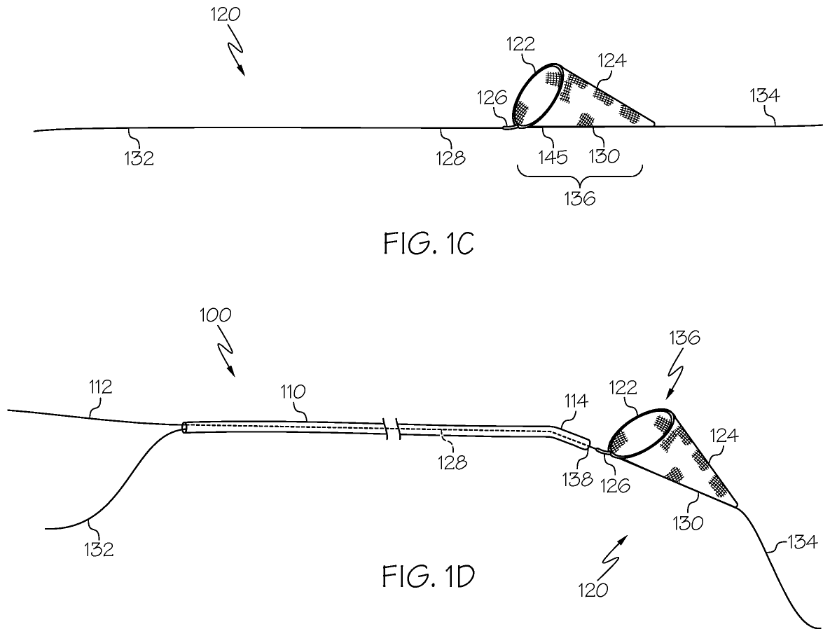

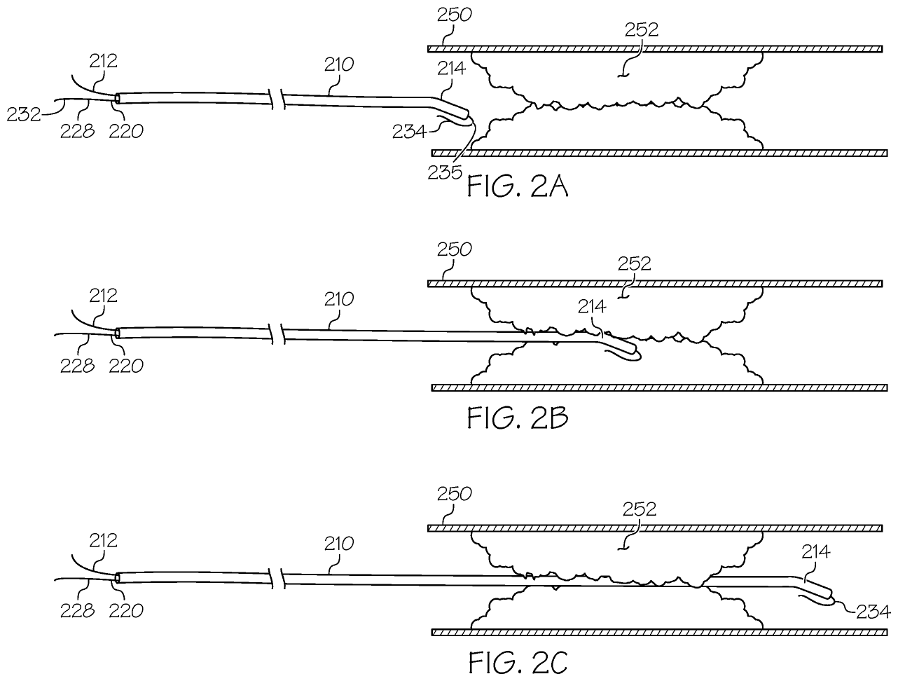

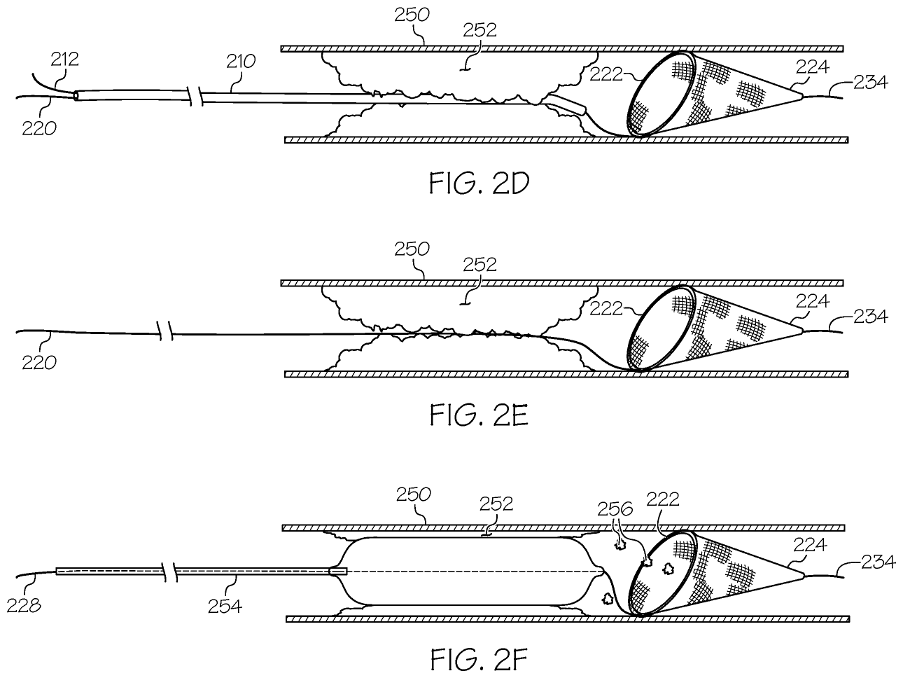

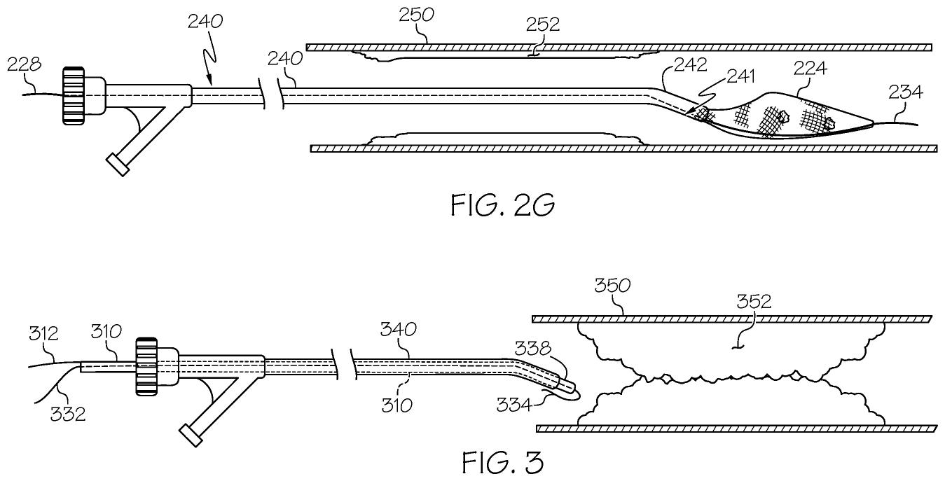

The figures herein follow a numbering convention in which the first digit or digits correspond to the figure number and the remaining digits identify an element or component in the figure. Similar elements or components between different figures may be identified by the use of similar digits. For example, 106 may reference element “6” in FIG. 1, and a similar element may be referenced as 206 in FIG. 2A. As will be appreciated, elements shown in the various embodiments herein can be added, exchanged, and/or eliminated so as to provide a number of additional embodiments of the present disclosure. In addition, the proportion and/or the relative scale of the elements provided in the figures are intended to illustrate certain embodiments of the present disclosure and should not be taken in a limiting sense.

FIG. 1A is a schematic diagram of a delivery catheter accordance with a number of embodiments of the present disclosure. In the example, the delivery catheter 110 can include a proximal wire 112 that is attached to the proximal end of the delivery catheter. In some examples, the distal portion of the delivery catheter 110 includes an angle section 114. In some examples, the delivery catheter 110 includes, optionally, an angled portion 114, a soft tip 138 and one or more marker bands 115, 115′. The delivery catheter can be used with any of the embolic protection devices disclosed herein. In some embodiments, delivery catheter 110 will not include proximal wire 112 and may, in some examples, include a y-adapter similar to y-adapter 143 shown in FIG. 1B.

FIG. 1B is a schematic diagram of a retrieval catheter in accordance with a number of embodiments of the present disclosure. In the example, retrieval catheter 140 comprises, optionally, one or more marker bands 141, an angled distal section 142, and a proximal y-adapter 143. In some embodiments, retrieval catheter 140 is sized such that it can be advanced over delivery catheter 110. In some embodiments, the retrieval catheter is advanced over proximal loop 122 to collapse the filter after use. The retrieval catheter can be used with any of the embolic protection devices disclosed herein.

FIG. 1C is a schematic diagram of the embolic protection apparatus 120 in accordance with a number of embodiments of the present disclosure. In this example, the embolic protection apparatus 120 can include a wire 128 and basket section or basket 136. In some embodiments, guidewire 128 has three sections: proximal section 132 which extends from the proximal end of the guidewire to about the location of expandable loop 122, distal section 134 that extends from the distal end of porous filter 124 to the end of the guidewire, and a basket section 130 that generally co-extends with loop 122 and porous filter 124 (filter section 136). The filter of basket section 136 is comprised of an expandable proximal loop 122 that is connected to a porous filter 124. Expandable loop 122 is connected to wire 128 via connector 126. This connection can be welded, soldered, made through the use of an adhesive, swaged, crimped, or the like. Following attachment of the wire to the expandable loop, the loop will be at an angle of about 75 to 90 degrees from the wire, or about 80 to 85 degrees from the wire. In some embodiments, the expandable loop 122 comprises a structural loop that provides support to the porous filter 124 material. In some embodiments, the expandable loop 122 comprises a structural hoop.

In some embodiments, the wire 128 comprises a first portion 132, a second portion 145 and a third portion 134. In some embodiments, each portion 132, 145, 134 comprises a length portion of the wire 128. In some embodiments, the second portion 145 is coextensive with at least a portion of the basket 136. In some embodiments, the third portion 134 is distal to the basket 136. In some embodiments, a cross-sectional area of the first portion 132 is greater than a cross-sectional area of the second portion 145.

FIG. 1D is a schematic diagram of a delivery catheter and embolic protection apparatus device in accordance with a number of embodiments of the present disclosure. In embodiments where the delivery catheter 110 and the embolic protection apparatus are provided separately, the physician will insert the embolic protection apparatus into the delivery catheter prior to use. In the example, the combined delivery catheter and embolic protection apparatus 100 can include a delivery catheter 110 and an embolic protection apparatus 120. In some examples, the distal portion of the delivery catheter 110 includes an angle section 114. In this example, the porous filter 124 and expandable loop 122 are shown in the expanded configuration. Shown in the figure is proximal wire 112. In some embodiments delivery catheter 110 includes a y-adapter (similar to 143) instead of wire 112.

In a number of embodiments, some of the sections of wire 128 have different diameters. In some embodiments, the diameter of the proximal section of the wire 132 is of a larger diameter than the basket section 130. Many of the commercially available devices for treating peripheral vascular disease are compatible with wires having a diameter of up to 0.035 inches. Many commercially available devices for treating coronary vascular disease are compatible with wires having a diameter up to 0.014 inches. While larger diameter wires provide more support than smaller diameter wires, the smaller diameter wires are generally more flexible and can more easily traverse tortuous anatomy. In some embodiments of the invention, the embolic protection apparatus 120 is positioned within delivery catheter 110. As will be explained later, the combined delivery catheter and embolic protection apparatus 120 are advanced together across a lesion. Crossing lesions, especially in tortuous anatomy, requires balancing many features, including pushability and flexibility. Thus, while a smaller diameter device will generally be more flexible, a larger diameter and/or stiffer device will have greater pushability. The diameter of the delivery catheter 110 is dependent on the minimum inside diameter needed to house the collapsed embolic protection apparatus 120 which includes wire 128. In the filter section 136, the effective diameter will comprise the diameter of the guide wire section 130 plus the space needed for collapsed proximal loop 122 and filter 124. In some embodiments, the competing needs of a large diameter wire needed for support and a small diameter profile needed for the delivery catheter is balanced by using a wire with a diameter over 0.030 inches for the proximal section 132 of the guidewire 128 and using a wire with a diameter less than 0.030 inches for the basket section of the wire, 130. In other embodiments, the diameter of the basket section of the wire 130 is less than 0.025 inches or less than 0.020 inches. In some embodiments, the diameter of the distal section of the wire 134 will be approximately equal to the diameter of wire 130. In some embodiments, the diameter of the distal section of the wire 134 will be equal to the diameter of the proximal section 132. In some embodiments, the diameter of the distal section of the wire 134 will be between about 0.010 and 0.018 inches. In some embodiments, the distal section of the wire 134 will comprise three subsections. The proximal subsection will have a wire diameter of greater than 0.30 inches, in some embodiments 0.035 inches. The middle subsection will have a wire diameter between 0.015 and 0.03 inches or between 0.02 and 0.03 inches, is some embodiments 0.18 inches. The distal subsection will have a wire diameter less than 0.02 inches or less than 0.015 inches, in some embodiments 0.014 inches.

FIG. 2A shows the delivery catheter 210 and embolic protection apparatus 220 system advanced in a lumen 250 to the proximal end of lesion 252. Lumen 250 can be any body lumen such as a blood vessel. Lesion 252 as shown in FIG. 2A is a total occlusion. As used herein, lesion can mean any lesion including a total occlusion, such as a chronic total occlusion, or a lumen that is occluded at least 50%, at least 75%, at least 90%, at least 95%, or more. The lesion can be formed from either plaque or thrombus or both. As shown in FIG. 2A, part of the distal wire section 234 of embolic protection apparatus 220 is located distally of the distal end of delivery catheter 210 and is looped back toward the proximal end. FIG. 2B shows the delivery catheter/embolic protection system advanced into the lesion and FIG. 2C shows the delivery catheter/embolic protection system advanced through the lesion 252. In some embodiments, the physician can use the radiopaque marker bands 115 of the delivery catheter to monitor the progress of the delivery catheter into and through the lesion. In embodiments where the delivery catheter has an angled distal section 214, the angled section can aid the physician in advancing the delivery catheter through tortuous vessels and through lesion 252. In some instances, the delivery catheter may come into contact with the wall of lumen 252. If the physician continues to advance catheter 210, it may advance into or through the adventitia, which is not a preferred clinical outcome. In the event that lesion 252 is the location of a previously implanted stent, the delivery catheter 210 could become lodged against a stent strut. The angled distal end 214 and the loop on the wire 234 allows the physician to rotate the catheter and/or the wire and to ‘point’ the distal end either back toward the center of the lumen and/or away from the stent strut.

In some embodiments, the third portion 234 of the wire extends distal to the catheter 210, comprises a curved portion 235 or bend, and overlaps with the catheter 210. In some embodiments, a tip of the wire is positioned adjacent to an external sidewall of the catheter 210.

FIG. 2D shows the delivery catheter 210 partially retracted such that expandable loop 222 and porous filter 224 are expanded. Expandable loop 222 expands into contact with lumen 250 and porous filter 224 opens. The diameter of expandable loop 222 is sized to be equal to or slightly larger than the diameter of lumen 250 so that it touches the inside surface of lumen 250. In some examples expandable loop 222 is radiopaque so that the physician can ensure that it has expanded into contact with the wall of the lumen. Expandable loop can be made more radiopaque through the use of platinum, tungsten, or gold markers crimped onto or applied to the loop. FIG. 2E shows delivery catheter 210 fully retracted leaving the filter section of embolic protection apparatus 220 positioned distal to the lesion 252. In FIG. 2F, a balloon catheter 254 has been advanced into lesion 252 by being advanced over wire 228 and expanded. While a balloon catheter is shown, any interventional device such as a balloon catheter, drug coated balloon catheter, stent delivery catheter, drug coated stent delivery catheter, thrombectomy catheter, or atherectomy catheter may be used to treat lesion 252. When balloon catheter 254 is advanced through lesion 252 and when it is expanded, embolic particles 256 may be released from lesion 252. The embolic particles can be thromboembolic (particles of thrombus) or particles of the plaque. By having expandable loop 222 and porous filter 224 in a delivery configuration, embolic protection apparatus 220 is able to capture emboli 256, protecting the downstream lumens.

After the intervention is complete, the user will retract the interventional device(s) leaving the embolic protection apparatus 220 in place. Lesion 252 has now been treated and the lumen 250 is substantially less occluded than it was prior to the intervention. As shown in FIG. 2G, the retrieval catheter 240 is then advanced over wire 228 and loop 222. The radiopaque marker 241 of the retrieval catheter and the radiopaque nature of loop 222 aids the physician in capturing the porous filter. In this example, porous filter is not totally inside retrieval catheter 240 as doing so could squeeze the embolic material 256 out through the pores of porous filter 224. In some embodiments, the loop 222 and filter 224 are completely within the retrieval catheter 240. In some embodiments, the retrieval catheter 240 can have an angled distal section 242. The angled section 242 can aid the physician in advancing the retrieval catheter through tortuous vessels and, in the event that lesion 252 included a previously implanted stent or if a stent was implanted during the interventional procedure, the angled section can be useful to the physician in advancing the retrieval catheter through the stent. In practice, a catheter can become lodged against a stent strut and the angled distal section 242 allows the physician to rotate the retrieval catheter 240 to direct the distal end away from the stent. The retrieval catheter 240 and embolic protection apparatus 220 can then be removed. In some embodiments, the delivery catheter 210 can be used to capture and remove the embolic protection apparatus.

FIG. 3 shows an embodiment of the invention where the retrieval catheter 340 is used with the delivery catheter 310 to provide additional backup support for the delivery catheter. In clinical settings where lumen 350 and lesion 352 are located distally of or within tortuous anatomy, the flexibility of the combined delivery catheter 310 and embolic protection apparatus 320 may be such that the physician cannot push or advance the delivery catheter through the challenging anatomy or lesion, for example: in tortuous vessels or chronic total occlusions. In these instances, the physician can advance the retrieval catheter 340 partially or completely over the delivery catheter 310 to provide backup support. With this support, the physician will be able to advance the entire system through the lumen to reach lesion 352. In some examples, the delivery catheter 310 may not be stiff enough for the physician to advance the delivery catheter through lesion 352. In these situations, the retrieval catheter can be advanced over the delivery catheter to provide backup support. The physician may advance only the delivery catheter 310 through lesion 352 or may also need to advance retrieval catheter 340 through lesion 352 in order to have a successful outcome.

In some embodiments shown, the wire section that extends with the filter section is positioned outside the porous filter. In these embodiments, the porous filter is attached to the expandable loop, and, in some embodiments, the distal end of the porous filter is attached to the wire. This attachment point may include a radiopaque marker. In other embodiments, the wire section that extends with the filter section is positioned inside the porous filter. In these embodiments, the porous filter is attached to the expandable loop, and, in some embodiments, the distal end of the porous filter is attached to the wire, preferably at the point where the wire exits the porous filter. This attachment point may include a radiopaque marker. For all the embodiments shown herein, both of these two configurations are applicable.

In some embodiments, a single sized embolic protection apparatus will be used in lumens with a wide range of diameters. For example, an embolic protection apparatus with an expandable loop diameter of 12 mm can be used in lumens ranging from 5 to 10 mm. When used in the smaller diameters, however, the porous filter material positioned near the expandable loop has a tendency to bunch up, creating a narrowed lumen of the basket that may prevent embolic particles to be able to enter the filter or, at a minimum, flow to the distal end of the filter. A flexible scaffolding can be used to eliminate this problem. The flexible scaffolding may be composed of one or more metallic wires that are attached on the proximal end to the device loop. On the distal end the one or more wires are anchored to a slidable ring which is positioned over the wire of the device. When catheter is placed over the embolic basket the slidable ring and the distal end of the scaffold will move distally enabling a smaller crimped diameter. When catheter is removed from over the basket the metallic wires expand within the basket preventing the basket from “bunching up” when the device is deployed in lumens with diameters smaller than the loop of the device.

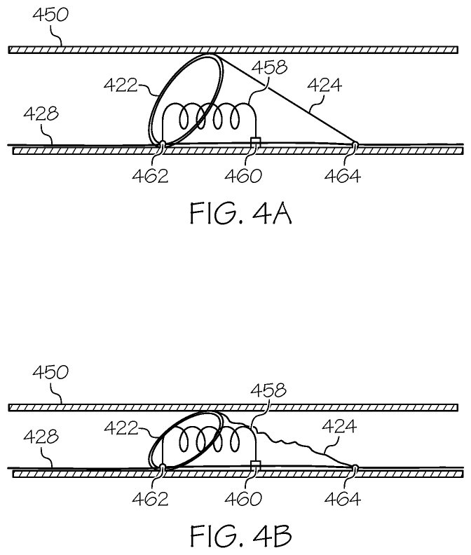

FIG. 4A shows an embolic protection apparatus positioned in a lumen with a size near the maximum indicated for the particular apparatus. Positioned in lumen 450, expandable loop 422 is shown attached to wire 428. As the diameter of lumen 450 is near the maximum indicated for the illustrated embolic protection apparatus, the expandable loop is nearly perpendicular to the longitudinal axis of lumen 450. Given the orientation of loop 422, porous filter 424 has an expanded conical shape. FIG. 4B shows an embolic protection apparatus positioned in a lumen with a size near the minimum indicated for the particular apparatus with the expandable loop 422 positioned away from a line perpendicular to the longitudinal axis of lumen 450. Given the orientation of loop 422, porous filter 424 has a tendency to bunch up near the expandable loop. Positioned within porous filter 424 is scaffolding 458. With a diameter just slightly less than the minimum indicated diameter for the embolic protection apparatus, scaffolding 458 holds porous filter 424 in a position that prevents the porous filter from blocking embolic particles from entering the filter. The proximal end of scaffolding 458 is attached to wire 428 near the proximal end of the porous filter and/or to expandable loop 422. The distal end of scaffolding 458 can be directly attached to wire 428 or can be attached to slide mechanism 460. Slide mechanism 460 allows the distal end of scaffolding 458 to slide along wire 428. When the embolic protection apparatus is positioned in the delivery catheter or retrieval catheter or in a smaller diameter lumen, the slide mechanism will slide distally. When the embolic protection apparatus is positioned in a larger diameter lumen, slide mechanism will slide proximally. In some embodiments, a radiopaque marker 464 is positioned at the distal end of the porous filter 424, where it is attached to wire 428.

In some embodiments, the slide mechanism is positioned on a length of wire that that extends in the filter section. In some embodiments, this reduced diameter section has a diameter or 0.010 to 0.020 inches. In some embodiments, the scaffolding has a spiral shape, but any shape such as a braid, a looped wire, or a random configuration will work as long as the scaffolding expands to hold open the porous filter and doesn't prevent embolic particles from entering the filter. The scaffolding can be made of nitinol, stainless steel, or any elastic or superelastic material.

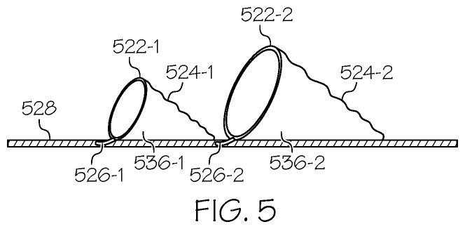

FIG. 5 is an embodiment of an embolic protection filter that comprises two filters on the same wire. FIG. 5 shows a proximal embolic protection filter which comprises expandable loop 522-1 and porous filter 524-1 positioned on wire 528. Positioned distal to this filter is a second, or distal, embolic protection filter which comprises expandable loop 522-2 and porous filter 524-2. This embodiment allows for a single device to treat a wide range of lumens. For example, the proximal filter may be sized to accommodate lumens between 4 and 7 mm. The distal filter may be sized to accommodate lumens between 8 and 11 mm. In this example, a single device will function in lumens from 4 to 10 mm. When used in smaller lumens, the proximal expandable loop 522-1 is in contact with the entire lumen wall and functions as the filter. When used in larger lumens, proximal loop 522-1 fully opens but does not contact the lumen wall while distal loop 522-2 is in contact with the entire lumen wall. In some embodiments, wire 528 will have a diameter of 0.035 inches from the proximal end to the proximal filter, and in the area that coextends with filter 524-1 and 524-2, wire 528 will have a diameter of 0.018 inches. In some embodiments, the wire distal of the distal filter will have a short segment of 0.035-inch wire, followed by a short segment of 0.018-inch wire, with wire 528 having a diameter of 0.014 inches at the distal tip.

In some embodiments, an embolic protection apparatus comprises a wire 528, a first basket 536-1 attached to the wire 528 and a second basket 536-2 attached to the wire 528. In some embodiments, the second basket 536-2 is larger than the first basket 536-1. In some embodiments, the first loop 522-1 comprises a cross-sectional area that is less than a cross-sectional area of the second loop 522-2.

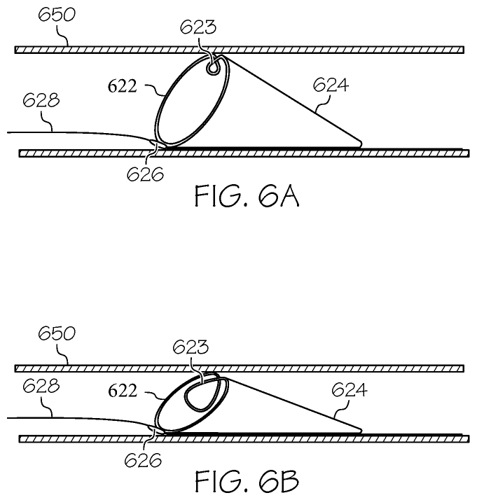

FIGS. 6A and 6B are an embodiment of an embolic protection filter where the area of the cavity defined by the expandable loop is variable. In some embodiments, the expandable loop comprises a primary and a secondary expandable loop. In this embodiment, expandable loop 622 includes a secondary loop, 623. As shown in FIG. 6A, when positioned within a large diameter lumen 650, secondary loop 623 assumes a small diameter. As shown in FIG. 6B, when positioned with a small diameter lumen 650, secondary loop 623 assumes a large diameter. This large diameter can be between 30% and 100% of the lumen diameter. When compressed into a delivery catheter, secondary loop 623 will enlarge as the expandable loop is forced into the delivery catheter. Upon delivery, secondary loop will be reduced, assuming a diameter which allows primary loop 622 to be in contact with the lumen wall. This arrangement allows for a single device to be used in lumens with a wide range of diameters. In some examples, this range can be from 4 mm to 11 mm. In some embodiments, wire 628 will have a diameter of 0.035 inches from the proximal end to the expandable loop 622. In the area that coextends with filter 624, wire 628 will have a diameter of 0.018 inches. Distal of the filter the wire 634 will have a short segment of 0.035-inch wire, followed by a short segment of 0.018-inch wire, with wire 628 having a diameter of 0.014 inches at the distal tip.

In some embodiments, the filter material 624 comprises a first attachment point to the expandable loop 622 located to a first side of the secondary loop 623. In some embodiments, the filter material 624 comprises a second attachment point to the expandable loop 622 located to a second side of the secondary loop 623. In some embodiments, the filter material 624 is not attached to the secondary loop 623 directly.

In some embodiments, an embolic protection apparatus comprises a first basket 536-1 and a second basket 536-2, for example as shown in FIG. 5, and either basket 536-1, 536-2 can comprise a secondary loop 623, for example as shown in FIG. 6A. In some embodiments, a larger basket (e.g. 536-2) comprises a secondary loop 623 and a smaller basket (e.g. 536-1) excludes a secondary loop 623.

The collapsible filters described herein may have a length of 2 cm to 7 cm. In some embodiments, the collapsible filter may have a length of 2.5 cm to 5 cm. In some embodiments where two filters are positioned on the wire, the proximal filter may have a length of 2 cm to 3 cm and the distal filter may have a length of 3 cm to 4 cm and the two filters can be positioned less than 1 cm apart. In some embodiments the length of the wire tip (the wire that is distal to the distal end of the basket) may be 10 cm 10 15 cm. In embodiments that have a segmented distal tip, each segment of the tip may have a length of 2 cm to 5 cm.

The device described herein may be used for two separate clinical indications. In many, but not all, cases both indications may exist. First, the device will serve as peripheral embolic protection device. Many endovascular procedures create unacceptable risk for peripheral embolizations, and many peripheral procedures are performed in presence of existing thrombus. The device will protect the patient from the risk of atheroemboli, and thromboemboli. Deployment of embolic protection basket distal to the lesion/thrombus will mitigate the risk of embolic complications during endovascular procedures. It's design and size can be tailored to peripheral arteries including iliac, femoral, popliteal, common carotid, subclavian and brachiocephalic trunk. Secondly, the devices design will allow the operator to easier cross chronic total occlusions of the above-mentioned arteries. In some cases, endovascular treatment of chronic total occlusions creates unacceptable risk of embolic complications, and the device described herein will allow the operator to treat CTOs in a safer, more intuitive, and expeditious manner. For example: presence of occluded peripheral graft with old thrombus creating the occlusion will always be associated with very high risk of embolic complications. The device described herein will significantly mitigate that risk.

In some embodiments, the delivery catheter and/or the retrieval catheter are shown as having an angled distal end. A straight distal end is also within the scope of this disclosure.

In some embodiments, a valve at the proximal end of the delivery catheter will have an outer diameter approximately equal to the diameter of the delivery catheter. In some embodiments, the delivery catheter, embolic protection apparatus, and retrieval catheter will be provided as a system or in a single pack. Prior to use, the physician will advance the delivery catheter over the distal protection apparatus until the only the distal section of the wire extends out of the distal end of the delivery catheter. The delivery catheter could have an attached or removable port to allow the physician to flush the catheter with saline or other appropriate fluid prior to use.

The apparatuses of this disclosure are useful in a number of clinical situations. Lesions, including thrombotic occlusions, in the superficial femoral artery (SFA), common femoral artery, popliteal artery, iliac artery, iliac bypasses, or femoropopliteal (fem-pop) bypasses may be treated with the apparatus described here. Vessels that extend off the aortic arch such as the brachiocephalic artery, the right and left common carotid artery, brachiocephalic trunk, brachial branch, and the left subclavian artery can be treated with these devices. The apparatus described herein is also useful in the venous system and can be used to treat lesions in the iliac, femoral, popliteal, brachial, subclavian, axillary, innominate veins, and in the Inferior Vena Cava as well as Superior Vena Cava. Depending on the clinical requirements, either a radial, brachial, subclavian, pedal, proximal tibial, or femoral access can be used.

While many of the examples herein show and describe the devices and methods being used and performed in the vascular system, the devices and methods have applicability to non-vascular lumens.

The retrieval catheter, delivery catheter, and embolic protection apparatus will be constructed from materials that are known in the art. The delivery and retrieval catheters may have a multilayer or single layer construction. In a multilayer construction, the catheter could have a polymer inside layer, surrounded by a support structure such as a metal braid which in turn is surrounded by an outer polymer layer. Either catheter could have a flexibility that is consistent over the length of the catheter or could have increased flexibility at the distal end. Alternatively, the catheters could be made from a single or multi-stream extrusion, with or without an internal support structure. When one or both of the delivery and retrieval catheters have one or more marker bands, the marker bands can be formed of any radiopaque material and be in the form of a ring attached to either the internal or external surface, embedded in the internal or external surface so that they are flush with the surface, embedded within the wall structure of the catheter, or be a radiopaque agent mixed with the plastic of the catheter. One or both of the delivery and retrieval catheters can have a distal tip that is softer and/or more flexible that the body of the catheter. The embolic protection filter wire can be constructed of superelastic materials, nitinol, stainless steel, cobalt-chromium-nickel-molybdenum-iron alloy, or cobalt-chrome alloy, or a combination thereof. In embodiments where the basket and/or distal section(s) of the wire have a lesser diameter than the proximal section, the smaller diameter can be achieved by grinding or milling of the wire or by attaching a smaller diameter wire to the distal end of a larger diameter wire. The expandable loop can be constructed from superelastic materials, nitinol, stainless steel, cobalt-chromium-nickel-molybdenum-iron alloy, or cobalt-chrome alloy, or a combination thereof.

The porous filter can be fabricated from a variety of different materials, such as, but not limited to, a woven or braided plastic or metallic mesh, a perforated polymer film, a shape memory material or mesh, combinations thereof, or other material that can be capable of capturing material within flowing blood, while allowing the blood to flow through the pores of the material. In some embodiments the porous filter comprises polytetrafluoroethylene (PTFE), expanded polytetrafluoroethylene (ePTFE), polyurethane, polyolefin elastomers, polyamides, nylons, polyethers, polyamide block ethers (PEBAX), polyesters, and/or co-polyesters. In some embodiments the filter material has a thickness of 0.001 inches (25 microns) and the material has an 85A Shore A Hardness. In some embodiments the porous filter can be woven or braided into a mesh and can be made from polyester, polyamide, polyurethane, nitinol, or stainless-steel filaments. The porous filter can have a variety of differently sized pores ranging from about 50 microns to about 200 microns, from about 60 microns to about 180 microns, or from about 75 microns to about 150 microns. For some applications, the pores can be sized up to 250 microns. The pores can have a variety of different configurations and can be circular, oval, polygonal, combinations thereof and the porous filter can include pores that are differently sized and configured. In practice, the pore size can vary as needed, so long as the pores are sized so that the pores do not compromise blood flow through the filter and collect emboli that can adversely affect downstream vessels. The porous filter can be coated with a hydrophilic coating, a heparinized coating, PTFE, silicone, combinations thereof, or other coatings. In some embodiments, the porous filter can be attached to the expandable loop by dip coating. In some embodiments, the porous filter can be attached to the expandable loop by being wrapped around the loop and then attached to itself, for example being sealed with heat or through an adhesive.

In some embodiments, the retrieval catheter will have a length of 120 to 140 cm with an outside diameter between 0.07 and 0.09 inches, preferably about 0.08 inches and with an inside diameter between 0.065 and 0.085 inches preferably about 0.07 inches. In some embodiments the delivery catheter will have a length of about 260 to 300 cm with an outside diameter between 0.06 and 0.08 inches preferably 0.06 inches and an inside diameter between 0.04 and 0.075 inches preferably 0.055 inches. In some embodiments, the catheter itself will have a length of 120 to 140 cm and the proximal wire will have a length of 120 to 160 cm. In some embodiments, the embolic protection device will have a length of 260 to 300 cm. In some embodiments, the wire will have a diameter of 0.035 inches. In embodiments where the basket and/or distal sections have a smaller diameter, they can have a diameter of 0.018 or 0.014 inches. In some embodiments where the delivery catheter is angled, the angled section 114 can be located 1 cm from the distal tip. In embodiments where the delivery catheter has one or more radiopaque markers, the distal marker can be located 1 cm from the distal end and the proximal band, if any, will be located 5 cm from the distal end. In some embodiments where the retrieval catheter is angled, the angled section 114 can be located 2 cm from the distal tip. In embodiments where the retrieval catheter has one or more radiopaque markers, the distal marker can be located 2 cm from the distal end. In embodiments where the distal section of the retrieval and/or delivery catheter are angled, they can be angled between 10 and 30 degrees away from the longitudinal axis of the catheter.

Although specific embodiments have been illustrated and described herein, those of ordinary skill in the art will appreciate that an arrangement calculated to achieve the same results can be substituted for the specific embodiments shown. For example, where the disclosure may show a system or a method with one example of a distal protection device, any distal protection device can be used including those disclosed herein. This disclosure is intended to cover adaptations or variations of one or more embodiments of the present disclosure. It is to be understood that the above description has been made in an illustrative fashion, and not a restrictive one. Combination of the above embodiments, and other embodiments not specifically described herein will be apparent to those of skill in the art upon reviewing the above description. The scope of the one or more embodiments of the present disclosure includes other applications in which the above structures and processes are used. Therefore, the scope of one or more embodiments of the present disclosure should be determined with reference to the appended claims, along with the full range of equivalents to which such claims are entitled.

In the foregoing Detailed Description, some features are grouped together in a single embodiment for the purpose of streamlining the disclosure. This method of disclosure is not to be interpreted as reflecting an intention that the disclosed embodiments of the present disclosure have to use more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

In some embodiments, a lesion crossing device, and/or a method comprising using a lesion crossing device, is described according to the following numbered paragraphs:

1. A system for providing access to a lumen comprising:
a delivery catheter having a lumen and a diameter;
an embolic protection apparatus having a proximal and a distal end, comprising a wire and a collapsible basket positioned near a distal end of the wire, the collapsible basket comprising an expandable loop attached to the wire and a porous filter attached at one end to the loop, the expandable loop comprising a material which enables the expandable loop to expand to an open configuration from a delivery configuration;
wherein the wire comprises a distal section of wire distal of the porous filter, a basket section of wire positioned between the expandable loop and a distal end of the porous filter, and a proximal section of wire proximal of expandable loop;
wherein the diameter of the wire in the proximal section is larger than the diameter of the wire in the basket section;
wherein, when the embolic protection apparatus is positioned within the delivery catheter, the distal section of the wire has a flexibility that allows it to be looped back over the distal end of the delivery catheter.
   2. The system of paragraph 1 further comprising:
a scaffold positioned within the porous filter.
   3. The system of paragraph 2 wherein the wire extends through the interior of the porous filter.
   4. The system of paragraph 3 wherein the distal end of the scaffold is secured to the wire.
   5. The system of paragraph 4 wherein the securement of the distal end of the scaffold to the wire comprises a loop and the loop can translate along the length of the wire.
   6. The system of paragraph 1 wherein the diameter of the proximal section of the wire is equal to or greater than 0.03 inches.
   7. The system of paragraph 6 wherein the diameter of the basket section of the wire is less than 0.03 inches.
   8. The system of paragraph 7 wherein the diameter of the basket section of the wire is equal to or less than 0.025 inches.
   9a. The system of paragraph 1 wherein the distal end of the delivery catheter is angled relative to a longitudinal axis of the delivery catheter.
   9b. The system of paragraph 1 wherein the wire distal section comprises a proximal subsection, a middle subsection, and a distal subsection, wherein the diameter of the wire in the proximal subsection is greater than 0.030 inches, the diameter of the wire in the proximal section is between 0.030 and 0.150 inches, and the diameter of the wire in the distal section is less than 0.015 inches.
   10. A method of accessing a lesion within an occluded lumen comprising:
advancing an embolic protection apparatus, comprising a wire and a collapsible basket, positioned in a delivery catheter, until a distal end of the wire and delivery catheter is positioned adjacent an lesion within a lumen, wherein the distal end of the wire extends past a distal end of the delivery catheter; wherein the distal end of the wire has a flexibility that allows is to loop back toward the proximal end of the delivery catheter; wherein the collapsible basket is positioned near the distal end of the wire and within the delivery catheter, the collapsible basket comprising an expandable loop and a porous filter, the expandable loop comprising a material which enables the expandable loop to expand to an open configuration from a delivery configuration;
advancing the delivery catheter and embolic protection apparatus through the lesion by applying force to the delivery catheter, with the distal end of the wire looped over a distal tip of the delivery catheter, until the distal end of the delivery catheter is distal of the lesion;
retracting the delivery catheter from over the collapsible basket and allowing the collapsible basket to assume an open configuration;
wherein the wire has a distal section of wire distal of the porous filter, a basket section of wire between a proximal and distal end of the collapsible basket, and a proximal section of wire proximal of expandable loop;
wherein the diameter of the wire in the major section is larger than the diameter of the wire in the basket section; and
wherein, when in the open configuration, the porous filter protects the lumen downstream of the lesion from embolic material.
   11. The method of paragraph 10 further comprising:
a retrieval catheter, wherein the retrieval catheter is advanced over the delivery catheter prior to advancing the delivery catheter and wire through the lesion, wherein the retrieval catheter is held stationary while the delivery catheter and the wire are advanced through the lesion, wherein the retrieval catheter provides backup support for the delivery catheter.
   12. The method of paragraph 11 wherein the retrieval catheter is retracted prior to retracting the delivery catheter to expand the collapsible filter.
   13. The method of paragraph 10 wherein a distal end of the delivery catheter is angled relative to a longitudinal axis of the delivery catheter.
   14. The method of paragraph 10 further comprising:
removing the delivery catheter so that the wire and the opened basket remains in the lumen with the collapsible basket positioned distal of the lesion in an expanded configuration;
advancing a treatment catheter, selected from the group consisting of balloon catheters, drug coated balloon catheters, stent delivery catheters, drug coated stent delivery catheters, thrombectomy catheters, and atherectomy systems, over the wire until the treatment catheter is positioned within the lesion;
treating the lesion with the treatment catheter;
removing the treatment catheter;
advancing a retrieval catheter over the wire until the expandable loop of the collapsible basket is positioned within the retrieval catheter; and
removing the retrieval catheter and wire.
   15. The method of paragraph 14 wherein both the expandable loop and the porous filter are positioned within the retrieval catheter prior to removing the retrieval catheter and wire.
   16. The method of paragraph 14 wherein the lumen is a blood vessel and wherein all the devices remain within the lumen in the area of the treatment during the treatment and are not advanced into or through the adventitia.
   17. The method of paragraph 13 wherein the lumen is a blood vessel and the lesion is located within a previously implanted stent and wherein the angled distal end of the delivery catheter allows the delivery catheter to be advanced through the lesion without catching the stent struts.
   18. An embolic protection apparatus comprising:
a wire with a collapsible basket positioned near a distal end of the wire;
the collapsible basket comprising an expandable loop attached to the wire and a porous filter attached at one end to the loop, the expandable loop comprising a material which enables the expandable loop to expand to an open configuration from a delivery configuration; and
a scaffold positioned within the porous filter, wherein the scaffold holds the porous filter open when the expandable loop is in the expanded configuration.
   19. The apparatus of paragraph 18 wherein the wire extends through the interior of the porous filter.
   20. The system of paragraph 19 wherein the distal end of the scaffold is secured to the wire.
   21. The system of paragraph 20 wherein the securement of the distal end of the scaffold to the wire comprises a loop and the loop can translate along the length of the wire.
   22. An embolic protection apparatus comprising:
a wire with a collapsible basket positioned near a distal end of the wire; and
the collapsible basket comprising an expandable loop attached to the wire and a porous filter attached at one end to the loop, the expandable loop comprising a material which enables the expandable loop to expand to an open configuration from a delivery configuration;
wherein the expandable loop comprises a primary loop and a secondary loop.
   23. The apparatus of paragraph 22 wherein the expandable loop is comprised of nitinol.
   24. The apparatus of paragraph 22 wherein the wire comprises a distal section of wire distal of the porous filter, a basket section of wire positioned between the expandable loop and a distal end of the porous filter, and a proximal section of wire proximal of expandable loop;
wherein the diameter of the wire in the proximal section is larger than the diameter of the wire in the basket section.
   25. The apparatus of paragraph 24 wherein the wire distal section comprises a proximal subsection, a middle subsection, and a distal subsection, wherein the diameter of the wire in the proximal subsection is greater than 0.030 inches, the diameter of the wire in the proximal section is between 0.030 and 0.150 inches, and the diameter of the wire in the distal section is less than 0.015 inches.
   26. The apparatus of paragraph 25 wherein the porous filter comprises polytetrafluoroethylene.
   27. The apparatus of paragraph 26 wherein the porous filter comprises pores with a diameter of 75 to 150 microns.
   28. An embolic protection apparatus comprising:
a wire with a plurality of collapsible baskets positioned near a distal end of the wire;
a first collapsible basket comprising a first expandable loop attached to the wire and a first porous filter attached at one end to the first loop;
a second collapsible basket, positioned distal to the first collapsible basket, comprising a second expandable loop attached to the wire and a second porous filter attached to the wire and a second porous filter attached at one end to the second loop.
   29. The apparatus of paragraph 28 wherein a diameter of the first loop is smaller than a diameter of the second loop.
   30. The apparatus of paragraph 29 wherein the first collapsible basket is configured to work in a lumen with a diameter of 5 to 7 mm and the second collapsible basket is configured to work in a lumen with a diameter of 8 to 11 mm.
   31. The apparatus of paragraph 30 wherein the wire comprises a distal section of wire distal of the distal filter, a basket section of wire positioned between the proximal expandable loop and a distal end of the distal filter, and a proximal section of wire proximal of the proximal expandable loop;
wherein the diameter of the wire in the proximal section is larger than the diameter of the wire in the basket section.
   32. The apparatus of paragraph 31 wherein the wire distal section comprises a proximal subsection, a middle subsection, and a distal subsection, wherein the diameter of the wire in the proximal subsection is greater than 0.030 inches, the diameter of the wire in the proximal section is between 0.030 and 0.150 inches, and the diameter of the wire in the distal section is less than 0.015 inches.
   33. The apparatus of paragraph 31 wherein the porous filter comprises polytetrafluoroethylene.
   34. The apparatus of paragraph 33 wherein the porous filter comprises pores with a diameter of 75 to 150 microns.

What is claimed is:

1. A device comprising:
an embolic protection apparatus comprising a wire and a basket attached to the wire;
the basket comprising a structural loop attached to a porous filter;
the wire comprising a first portion and a second portion, the first portion proximal to the basket, the second portion coextensive with at least a portion of the basket, a cross-sectional area of the first portion greater than a cross-sectional area of the second portion.

2. The device of claim 1, comprising a catheter comprising a lumen, the basket positioned in the lumen.

3. The device of claim 2, the wire comprising a third portion distal to the basket, the third portion extending distally from the catheter, comprising a bend and overlapping the catheter.

4. The device of claim 1, the basket comprising a first basket, the embolic protection apparatus comprising a second basket attached to the wire.

5. The device of claim 4, the second basket larger than the first basket.

6. The device of claim 1, the basket comprising a scaffold arranged to support the porous filter.

7. The device of claim 6, the scaffold attached to the wire.

8. The device of claim 1, the structural loop comprising a secondary loop.

9. The device of claim 1, wherein a cross-sectional area of an aperture defined by the structural loop is variable.

10. The device of claim 1, the wire comprising a third portion distal to the basket, a cross-sectional area of the third portion greater than the cross-sectional area of the second portion

11. A device comprising:
an embolic protection apparatus comprising a wire, a first basket attached to the wire and a second basket attached to the wire;
the first basket comprising a first loop attached to a first porous filter;
the second basket comprising a second loop attached to a second porous filter.

12. The device of claim 11, wherein the second basket is larger than the first basket.

13. The device of claim 11, wherein an aperture defined by the second loop is larger than an aperture defined by the first loop.

14. The device of claim 11, the wire comprising a first portion and a second portion, the first portion proximal to the first basket, the second portion coextensive with at least a portion of the first basket, a cross-sectional area of the first portion greater than a cross-sectional area of the second portion.

15. The device of claim 11, the second loop comprising a secondary loop.

16. The device of claim 11, comprising a catheter comprising a lumen, the first basket positioned in the lumen, the second basket positioned in the lumen.

17. A device comprising:
an embolic protection apparatus comprising a wire and a basket attached to the wire;
the basket comprising a structural loop attached to a porous filter, the structural loop comprising a secondary loop.

18. The device of claim 17, wherein a cross-sectional area of the structural loop changes as a size of the secondary loop changes.

19. The device of claim 17, the structural loop attached to the wire at an attachment point, the secondary loop located opposite the attachment point.

20. The device of claim 17, the wire comprising a first portion and a second portion, the first portion proximal to the basket, the second portion coextensive with at least a portion of the basket, a cross-sectional area of the first portion greater than a cross-sectional area of the second portion.
